(19) United States
(12) Patent Application Publication
(10) Pub. No.: US 2005/0203605 A1
(43) Pub. Date: Sep. 15, 2005

(54) Radially crush-resistant stent

(75) Inventor: Mark Jeffrey Dolan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/079,956

(22) Filed: Mar. 15, 2005

Related U.S. Application Data
(60) Provisional application No. 60/553,208, filed on Mar. 15, 2004.

(51) Int. Cl. A61F 2/06

(52) U.S. Cl. 623/1.11

(57) ABSTRACT

A system for treating a vascular condition includes a catheter and a stent coupled to the catheter. The stent includes a stent framework having at least one stent segment with a plurality of interconnected struts and crowns and at least one stiffening ring having a plurality of ring segments connected between circumferentially adjacent crowns of the stent segment. The stiffening ring is oriented circumferentially about a longitudinal axis of the stent framework when the stent is deployed. A stent and a method of treating a vascular condition are also disclosed.

### RELATED APPLICATIONS

This application claims the benefit of U.S. Provisional Patent Application 60/553,208 filed Mar. 15, 2004.

### FIELD OF THE INVENTION

This invention relates generally to biomedical stents and valves. More specifically, the invention relates to a stent having an adapted stent framework to increase radial stiffness, reduce radial crush, reduce deployment recoil, and minimize overexpansion, while minimizing length changes during expansion.

### BACKGROUND OF THE INVENTION

Biomedical stents may be implanted and deployed within the human body to reinforce blood vessels or other vessels as part of surgical procedures for enlarging and stabilizing body lumens, or to support bioprosthetic valves implanted within the circulatory system. With generally open tubular structures of metallic or polymeric material, endovascular stents typically have apertured or lattice-like walls, and can be either balloon expandable or self-expanding. A stent is usually deployed by mounting the stent on a balloon portion of a balloon catheter, positioning the stent in a body lumen, and expanding the stent by inflating the balloon. The balloon is then deflated and removed, leaving the stent in place.

There is increasing evidence that stent design influences angiographic restenosis and clinical outcomes. An ideal stent possesses a low profile, good flexibility to navigate tortuous vessels, adequate radiopacity, low recoil, sufficient radial strength, minimal shortening upon expansion, and high scaffolding ability. Favorable clinical outcomes are influenced by the material composition of the stent and any surface coatings, as well as the stent geometry and thickness that affect the expansion of the stent and reduce the recoil of the stent. A desirable endovascular stent provides an ease of delivery and necessary structural characteristics for vascular support, as well as long-term biocompatibility, antithrombogenicity, and antiproliferative capabilities.

Some of the latest stent designs include coatings from which one or more drug agents are eluted. Stents can be coated with protective materials such as polymers to improve biocompatibility and prevent corrosion, and with bioactive agents to help reduce tissue inflammation, thrombosis and restenosis at the site being supported by the stent.

Stents may be used along with prosthetic tissue valves in procedures for replacing diseased and malfunctioning heart valves. For example, a stent can hold an artery open and support a valved pulmonary conduit that is used to reconstruct a blood pathway from the right ventricle of the heart to a patient's lungs. Medical procedures also use stents to provide structure and protection for aortic and mitral bioprostheses. A stented tissue valve may include a frame on which the valve is mounted to support the leaflets that control the directional flow of blood. Bovine jugular veins containing an integral valve can be used for such conduits.

An elastically collapsible and stent-mounted valve is described in “Valve Prosthesis for Implantation in the Body,” Andersen et al., U.S. Pat. No. 6,168,614 granted Jan. 2, 2001, and “System and Method for Implanting Cardiac Valves,” Andersen et al., U.S. Pat. No. 5,840,081 granted Nov. 24, 1998. The catheter-deployed valve prosthesis comprises a stent made from an expandable cylindrical thread structure, which can be compressed around a balloon means and expanded at a treatment area such as against the wall of the aorta.

Area of concerns for stent deployment, particularly those including valve prostheses, involve the need to prevent overexpansion of the stent, as well as to minimize stent recoil or spring-back, which may range from 3% to 20% in currently available stents. Stents are susceptible to radial crush and insufficient radial elasticity.

Accordingly, what is needed is an improved stent design providing resistance to overexpansion, minimization of recoil, optimal coverage of the vessel wall, and suitable flexibility while maintaining mechanical integrity during the deployment of the stent. The improved stent should have high radial strength to resist vessel recoil and excellent deliverability in tortuous or challenging anatomy. Additionally, an associated system and method for treating a vascular condition are needed for preventing undesirable radial crush or insufficient radial stiffness of a stent.

### SUMMARY OF THE INVENTION

One aspect of the invention provides a system for treating a vascular condition, which includes a catheter and a stent coupled to the catheter. The stent includes a stent framework having at least one stent segment with a plurality of interconnected struts and crowns and at least one stiffening ring having a plurality of ring segments connected between circumferentially adjacent crowns of the stent segment. The stiffening ring is oriented circumferentially about a longitudinal axis of the stent framework when the stent is deployed.

Another aspect of the invention is a stent including a stent framework having at least one stent segment with a plurality of interconnected struts and crowns, and at least one stiffening ring having a plurality of ring segments connected between circumferentially adjacent crowns of the stent segment. The stiffening ring is oriented circumferentially about a longitudinal axis of the stent framework when the stent is deployed.

Another aspect of the invention is a method of treating a vascular condition. A stent having a bioprosthetic valve is delivered to a targeted region via a catheter, and expanded to deploy the bioprosthetic valve. At least one stiffening ring of the stent is formed as the stent is expanded.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 illustrates a system for treating a vascular condition, in accordance with one embodiment of the current invention;

FIG. 2 illustrates a stent framework having one stent segment and a plurality of ring segments connected between circumferentially adjacent crowns of the stent segment, in accordance with one embodiment of the current invention;

FIG. 3 illustrates an expanded stent as described with respect to FIG. 2, in accordance with one embodiment of the current invention;

FIG. 4 illustrates a portion of a stent having a plurality of ring segments connected between circumferentially adjacent crowns of a stent segment, in accordance with one embodiment of the current invention;

FIG. 5 illustrates an expanded portion of a stent as described with respect to FIG. 4, in accordance with one embodiment of the current invention;

FIG. 6 illustrates a pattern for cutting a stent including a plurality of stent segments with a plurality of ring segments connected between circumferentially adjacent crowns of the stent segments, in accordance with one embodiment of the current invention;

FIG. 7 illustrates a stent including a plurality of stent segments with two end segments having no stiffening rings and a bioprosthetic valve positioned and attached within a central lumen of the stent framework, in accordance with one embodiment of the current invention; and

FIG. 8 is a flow diagram of a method of treating a vascular condition, in accordance with one embodiment of the current invention.

### DETAILED DESCRIPTION OF THE PRESENTLY PREFERRED EMBODIMENTS

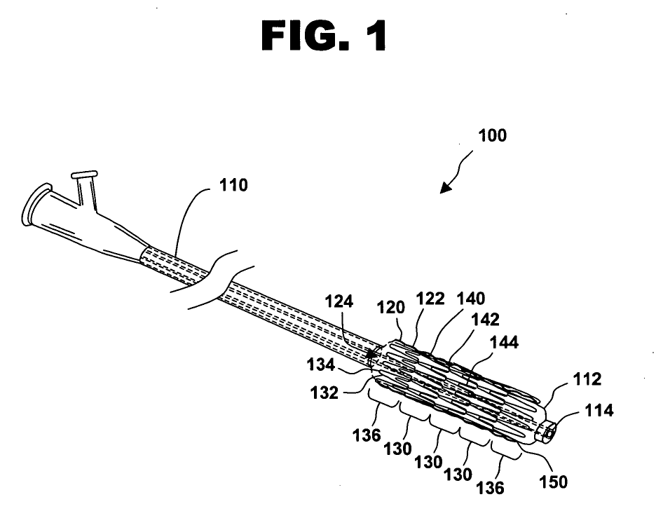

FIG. 1 illustrates a system for treating a vascular condition, in accordance with one embodiment of the present invention. Vascular condition treatment system 100 includes a catheter 110 and a stent 120 coupled to catheter 110. Stent 120 includes a stent framework 122 having at least one stent segment 130 with a plurality of interconnected struts 132 and crowns 134 and at least one stiffening ring 140 having a plurality of ring segments 142 connected between circumferentially adjacent crowns 134 of stent segment 130. Stent segments 130 are sinusoidally shaped, continuously formed in a loop or ring with smooth, rounded corners referred to as crowns 134 at each bend, and substantially straight segments in between crowns 134 referred to as struts 132. In one example, struts 132 and crowns 134 have a nominally uniform length and radius, respectively.

In one example, expandable stent 120 is configured to support a vascular lumen. Stent 120 is comprised of multiple stent segments 130 with sinusoidal patterns. A series of larger sinusoidal patterns with interconnected struts 132 and crowns 134 support the vascular lumen, while a series of smaller sinusoidal patterns form ring segments 142 that support the larger patterns upon deployment of stent 120. The larger sinusoidal patterns are connected cylindrically to form stent segments 130. Additional stent segments 130 and end segments 136 may be connected together to provide additional stent length. The smaller sinusoidal patterns are also connected circumferentially, with each ring segment 142 attached near crowns 134 of the larger sinusoidal patterns. The smaller sinusoidal pattern resides within the larger pattern, and is extended to form stiffening ring 140 when stent 120 is expanded.

As stent 120 is expanded and deployed, struts 132 and crowns 134 bend and straighten as the stent is enlarged diametrically, with minimal contraction extensionally.

One or more stiffening rings 140 are oriented circumferentially about a longitudinal axis of stent framework 122 when stent 120 is deployed. Stiffening ring 140, sometimes referred to as a lockout ring, comprises a plurality of ring segments 142 connected between circumferentially adjacent crowns 134. When in a compressed state, for example, each ring segment 142 has two struts 132 and crown 134. The length of struts 144 of ring segments 142 is less than the length of corresponding struts 132 of stent segments 130. When enlarged, ring segments 142 are substantially straightened to provide a higher degree of radial stiffness compared to that of struts 132 and crowns 134 alone. When stent 120 is expanded to prop open a vessel, ring segments 142 form an undulating ring-like shape that is stronger than the sinusoidal shape of struts 132 and crowns 134. Although fully extended ring segments 142 provide the largest amount of radial stiffness, ring segments 142 with a ring segment angle of up to approximately thirty degrees provides significant additional radial stiffness to minimize or eliminate deployment recoil.

Stiffening rings 140 minimize over expansion of stent framework 122 while stent 120 is being deployed. When ring segments 142 are straightened as balloon 112 is inflated, expansion of stent framework 122 becomes restricted. The deployed stent diameter may be controlled by the lengths of ring segments 142, which may be varied along the length of stent 120. For example, stent 120 comprising multiple lockout or stiffening rings 140 may have a funnel shape, a tapered shape, or an outwardly expanding shape. In another example, stent 120 may have an asymmetric shape when deployed, whereby one end of stent 120 is restricted to a prescribed stent diameter and the other end of stent 120 is allowed to expand and flare out unimpeded by any stiffening ring 140. In another example, end segments 136 have stiffening rings 140 corresponding to different stent diameters when stent 120 is deployed. Thus, the stiffening or lock-out rings 140 prevent localized over expansion, allowing other segments to expand to a larger diameter. The ring 140 creates a restriction and the over expanded segments are a funnel, which can improve sealing over the ostia or minimize stent migration.

When expanded and deployed within a vessel of a body, stiffening rings 140 of stent 120 reduce the tendency of inwardly directed forces from walls of the vessel to radially distort or radially crush deployed stent 120. Substantially formed stiffening rings 140 reduced the deployment recoil of stent 120 that may occur when stent 120 is expanded with an inflatable balloon 112. Once stent 120 is expanded and ring segments 142 are straightened to form stiffening rings 140, further expansion of stent 120 becomes more difficult because of the increased radial stiffness. Further increases of the stent diameter are restricted, in part due to the increased radial stiffness of formed stiffening rings 140 that limit a deployment diameter of stent 120.

Stiffening rings 140 may have substantially uniform length to provide diametric uniformity to stent 120 when expanded. Alternatively, variations in the lengths of ring segments 142 allow stiffening rings 140 to have variations in diameter with position along the length of stent 120 to form, for example, a funnel-shaped stent or a stent with enlarged or flared ends. Stiffening rings 140 may be omitted from end segments 136 to allow end segments 136 of deployed stent 120 to flare, which may improve fluid flow characteristics.

Catheter 110 may include an inflatable balloon 112 used to expand stent 120. Alternatively, catheter 110 may include a sheath that is removed or retracts to allow expansion of stent 120 in a self-expanding version as is known in the art. Catheter 110 of an exemplary embodiment of the present invention includes balloon 112 that expands and deploys stent 120 within a vessel of the body. Stent 120 is coupled to catheter 110, and may be deployed by pressurizing balloon 112 coupled to the stent and expanding stent 120 to a prescribed diameter. A flexible guidewire (not shown) traversing through a guidewire lumen 114 inside catheter 110 helps guide stent 120 to a treatment site, and once stent 120 is positioned, balloon 112 is inflated by pressurizing a fluid such as a contrast fluid that flows through a tube inside catheter 110 and into balloon 112. Stent 120 is expanded by balloon 112 until the desired diameter is reached, and then the contrast fluid is depressurized or pumped out, separating balloon 112 from deployed stent 120.

Stent framework 122 may include one or more end segments 136 with stiffening rings 140. Alternatively, stent framework 122 may include one or more end segments 136 without stiffening rings 140.

Stent framework 122 may include a polymeric base or a metallic base such as stainless steel, nitinol, tantalum, MP35N alloy, a cobalt-based alloy, platinum, titanium, a suitable biocompatible alloy, a suitable biocompatible material, and combinations thereof.

Selected crowns 134 of one stent segment 130 may be connected to corresponding crowns 134 on an adjacent stent segment 130. Crowns 134 of stent segment 130 are connected to corresponding crowns 134 on an adjacent stent segment 130 with, for example, a welded joint. Alternatively, crowns 134 of stent segment 130 may be connected to corresponding crowns 134 on an adjacent stent segment 130 with a molded joint, such as when stent 120 is formed from polymeric materials by a molding or casting process.

In one form of manufacturing, stent framework 122 is cut from a tube with a laser or a water-jet cutting tool. For example, an extruded tube of stainless steel, nitinol or other suitable metal is mounted on a mandrel and cut with a laser, then treated to achieved the desired finish. In another form of manufacturing using one or more stent segments 130 formed from shaping and bending wire, crowns 134 of one stent segment 130 may be connected to corresponding crowns 134 of an adjacent stent segment 130 with one or more welded joints. In another form of manufacturing using polymeric materials, crowns 134 of one stent segment 130 may be connected to corresponding crowns 134 of an adjacent stent segment 130 with one or more molded joints. The stent framework is formed from metal or polymers with a cast or a mold, the cast or mold having molded joints between connected crowns 134.

Stent 120 with one or more stent segments 130 and one or more stiffening rings 140 may be manufactured to an appropriate length and diameter to be inserted and deployed at various locations within the body. Stent 120 with or without drug-polymer coating 150 may be used, for example, as a cardiovascular stent, a peripheral stent, an abdominal aortic aneurysm stent, a cerebral stent, a carotid stent, an endovascular stent, an aortic valve stent, or a pulmonary valve stent. Insertion of stent 120 into a vessel of the body helps treat, for example, heart disease, various cardiovascular ailments, and other vascular conditions. Catheter-deployed stent 120 typically is used to treat one or more blockages, occlusions, stenoses, or diseased regions in the coronary artery, femoral artery, peripheral arteries, and other arteries in the body. Treatment of vascular conditions involves the prevention or correction of various ailments and deficiencies associated with the cardiovascular system, the cerebrovascular system, urinogenital systems, biliary conduits, abdominal passageways and other biological vessels within the body. Generally tubular in shape with flexibility to bend along a central axis, stent 120 expands with the help of a stent deployment balloon 112 or self-expands when released for a self-expanding version.

A bioprosthetic valve, not shown, may be attached to stent framework 122 and positioned within a central lumen 124 of stent framework 122. The bioprosthetic valve comprises, for example, a bovine jugular valve from a bovine jugular vein. Alternatively, a bioprosthetic valve such as a bovine valve, a porcine valve, an ovine valve, or an equine valve may be harvested or extracted from various mammals.

To reduce the chance of restenosis or other medical conditions from occurring in the vicinity of the stent, stent 120 may include a drug-polymer coating 150 disposed on stent framework 122 of stent 120. An exemplary coating material, such as a polymeric matrix and therapeutic compounds in a solvent, may be applied to a stent by dipping, spraying, paint, or brushing techniques, as is known in the art.

Drug-polymer coating 150 may be disposed on stent framework 122 to provide desired therapeutic properties. An exemplary drug-polymer coating 150 comprises one or more therapeutic agents that are eluted with controlled time delivery after the deployment of stent 120 within the body. Therapeutic agents are capable of producing a beneficial effect against one or more conditions including coronary restenosis, cardiovascular restenosis, angiographic restenosis, arteriosclerosis, hyperplasia, and other diseases or conditions.

Drug-polymer coating 150 includes, for example, a therapeutic agent such as rapamycin, a rapamycin derivative, a rapamycin analogue, an antirestenotic drug, an anti-cancer agent, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and combinations thereof.

Incorporation of a drug or other therapeutic agents into drug-polymer coating 150 allows, for example, the rapid delivery of a pharmacologically active drug or bioactive agent within twenty-four hours following the deployment of stent 120, with a slower, steady delivery of a second bioactive agent over the next three to six months. The thickness of drug-polymer coating 150 may extend, for example, between 1.0 microns and 200 microns or greater in order to provide sufficient and satisfactory pharmacological benefit.

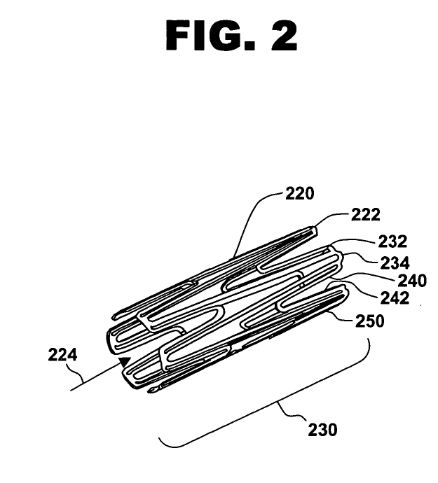

FIG. 2 illustrates a stent framework having one stent segment and a plurality of ring segments connected between circumferentially adjacent crowns of the stent segment, in accordance with one embodiment of the present invention. Stent 220 includes stent framework 222 having one stent segment 230 with a plurality of interconnected struts 232 and crowns 234. Two stiffening rings 240 having a plurality of ring segments 242 are connected between circumferentially adjacent crowns 234 of stent segment 230. Stiffening ring 240 is oriented circumferentially about a longitudinal axis through a central lumen 224 of stent framework 222 when stent 220 is deployed. Shown in a compressed or unexpanded state, ring segments 242 are located near each end of single-segment stent 220.

A bioprosthetic valve, not shown, may be positioned within a central lumen 224 of stent framework 222 and attached to stent 220 using, for example, sutures or stitches.

A drug-polymer coating 250 with one or more therapeutic agents may optionally be disposed on stent framework 222.

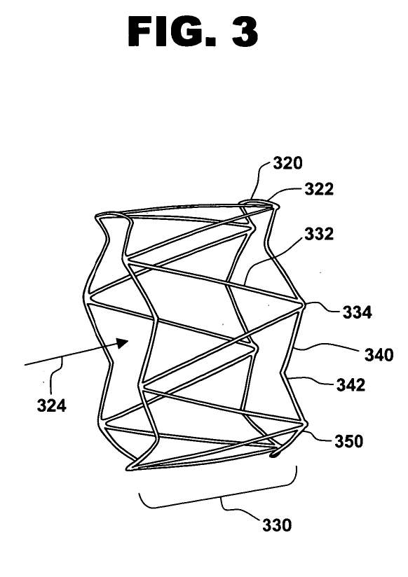

FIG. 3 illustrates an expanded stent as described with respect to FIG. 2, in accordance with one embodiment of the present invention. Similar elements are numbered with an increment of 100 to aid in clarity. Stent 320 with stent framework 322 having a single stent segment 330 is enlarged, for example, with an inflatable balloon to support the walls of a vessel and to allow the flow of fluid through a central lumen 324. Stent segment 330 has a plurality of interconnected struts 332 and crowns 334, with stiffening rings 340 comprising ring segments 342 connected between circumferentially adjacent crowns 334. Stiffening rings 340 are formed when stent framework 322 is expanded and ring segments 342 are substantially straightened. Substantial radial stiffness is achieved when ring segments 342 are straightened, although appreciable radial stiffness to reduce recoil and improve radial crush characteristics occurs when the angles of ring segments 342 are as large as twenty to thirty degrees or more from a fully straightened configuration. Stiffening rings 340 of stent 320 reduce radial crush and deployment recoil, limit the deployed diameter of stent 320, and increase the radial stiffness when formed.

An optional drug-polymer coating 350 with one or more therapeutic agents may be disposed on stent framework 322. A bioprosthetic valve (not shown) may be positioned within central lumen 324 of stent framework 322 and attached to stent 320 using, for example, sutures or stitches.

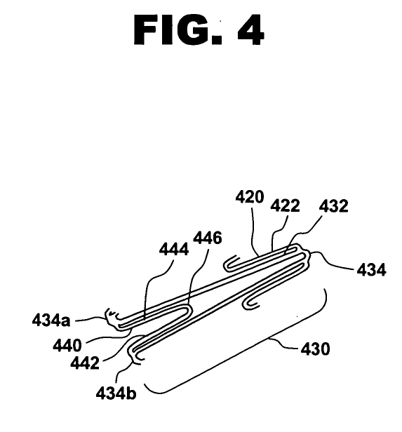

FIG. 4 illustrates a portion of a stent 420 with interconnected struts 432 and crowns 434, and with a plurality of ring segments connected between circumferentially adjacent crowns 434 of a stent segment 430, in accordance with one embodiment of the present invention. Ring segments 442 of stiffening ring 440 may have associated ring segment struts 444 and ring segment crowns 446 that are pulled substantially straight when stent 420 is expanded. Ring segments 442 may be connected between circumferentially adjacent crowns 434a and 434b of stent framework 422.

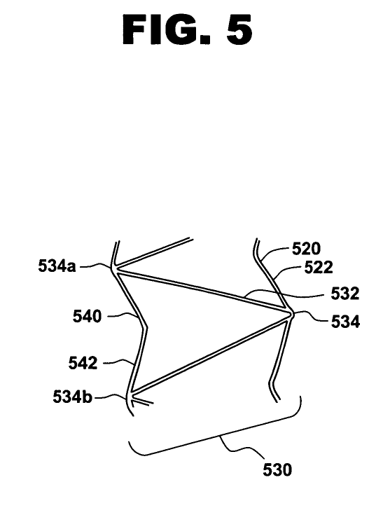

FIG. 5 illustrates an expanded portion of a stent as described with respect to FIG. 4, in accordance with one embodiment of the present invention. The numbers of similar elements in previous figures are incremented by 100 to aid clarity. Stent 520 with stent framework 522 having struts 532 and crowns 534 of a stent segment 530 is diametrically enlarged with minimal foreshortening of the stent length. As stent 520 is enlarged, stiffening ring 540 comprising a plurality of ring segments 542 between circumferentially adjacent crowns 534a and 534b are substantially straightened to increase the radial stiffness of stent 520.

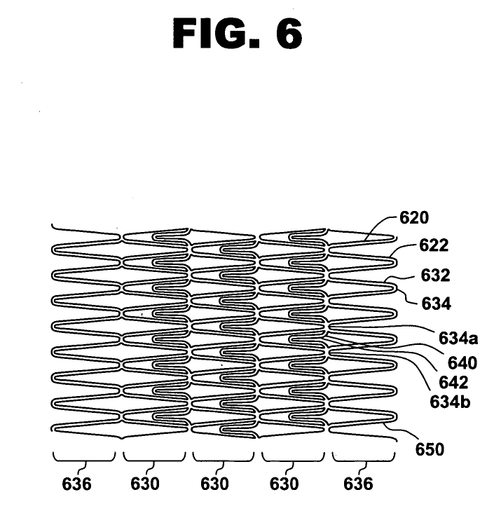

FIG. 6 illustrates a pattern for cutting a stent including a plurality of stent segments with a plurality of ring segments connected between circumferentially adjacent crowns of the stent segments, in accordance with one embodiment of the present invention. Selected crowns 634 of stent segments 630 are connected to corresponding crowns 634 of adjacent stent segments 630. Additionally, selected crowns 634 of end segments 636 are connected to corresponding crowns 634 on adjacent stent segments 630. Stent segments 630 and end segments 636 include one or more stiffening rings 640 comprising a plurality of ring segments 642 connected between circumferentially adjacent crowns 634a and 634b. Stiffening rings 640 are formed when stent framework 622 of stent 620 with struts 632 and crowns 634 is enlarged.

A bioprosthetic valve, not shown, may be positioned within a central lumen of stent 620. A drug-polymer coating 650 may be disposed on stent framework 622 of stent 620.

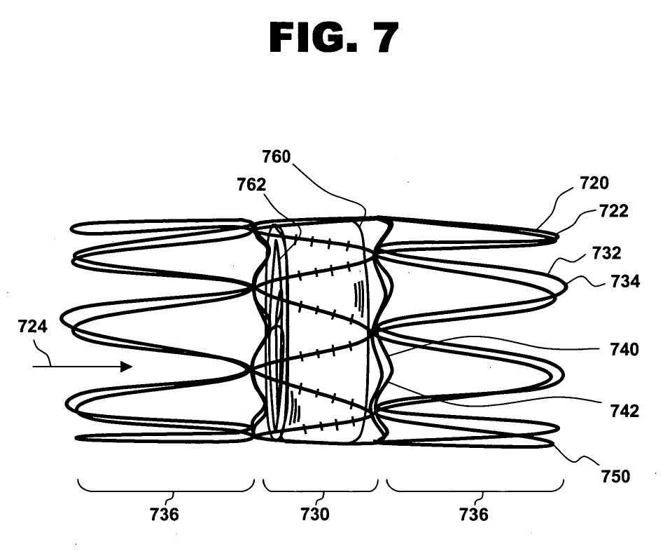

FIG. 7 illustrates a stent including a plurality of stent segments with two end segments having no stiffening rings and a bioprosthetic valve positioned and attached within a central lumen of the stent framework, in accordance with one embodiment of the present invention. Stent 720 with stent framework 722 comprises a stent segment 730 with interconnected struts 732 and crowns 734. Two end segments 736 are connected to stent segment 730 at selected crowns 734. Stiffening rings 740 may be included or omitted from end segments 736. When stent 720 is expanded, two stiffening rings 740 are formed from ring segments 742 connected between circumferentially adjacent crowns 734. A drug-polymer coating 750 may be disposed on stent framework 722 of stent 720. A bioprosthetic valve 760 such as a bovine jugular valve is positioned within a central lumen 724 of stent framework 722 and attached thereto. Valve leaflets 762 open and close to control the direction of fluid flow through valve 760.

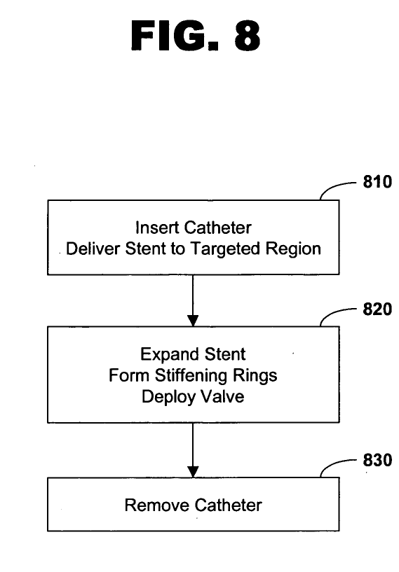

FIG. 8 is a flow diagram of a method of treating a vascular condition, in accordance with one embodiment of the present invention. The method includes various steps to deploy a stent having one or more stiffening rings that form when the stent is enlarged.

A stent including one or more stent segments and at least one stiffening ring with a plurality of ring segments is provided. Each stent segment includes a plurality of interconnected crowns and struts. One or more end segments may also be included. The stent segments, end segments and stiffening ring segments are formed, for example, by cutting a tube with a laser or a water jet. The initial stent material may include, for example, stainless steel, nitinol, tantalum, MP35N alloy, a cobalt-based alloy, platinum, titanium, a suitable biocompatible alloy, a suitable biocompatible material, or combinations thereof. The stent framework is cleaned using, for example, degreasers, solvents, surfactants, de-ionized water or other cleaners, as is known in the art.

The stent may have a drug-polymer coating applied to the stent framework. An exemplary drug polymer that includes a polymeric matrix and one or more therapeutic compounds is mixed with a suitable solvent to form a polymeric solution, and is applied using an application technique such as dipping, spraying, paint, or brushing. During the coating operation, the drug-polymer adheres to the stent framework and any excess drug-polymer solution may be removed, for example, by being blown off. In order to eliminate or remove any volatile components, the polymeric solution may be dried at room temperature or at elevated temperatures under dry nitrogen or another suitable environment. A second dipping and drying step may be used to increase the thickness of the drug-polymer coating, the thickness ranging between 1.0 microns and 200 microns or greater in order to provide sufficient and satisfactory pharmacological benefit.

The drug-polymer coating may be treated, for example, by heating the drug-polymer coating to a predetermined temperature to drive off any remaining solvent or to effect any additional crosslinking or polymerization. The drug-polymer coating may be treated with air drying or low-temperature heating in an air, nitrogen, or other controlled environment.

The drug-polymer coating may be applied before or after rolling the stent framework down to a desired diameter before insertion into the body.

The coated or uncoated stent may be integrated into a system for treating vascular conditions such as heart disease by coupling the stent to the catheter. Exemplary finished stents are reduced in diameter, placed into the distal end of the catheter, and formed, for example, with an interference fit that secures the stent onto the catheter. Radiopaque markers may be attached to the stent or catheter to aid in the placement of the stent within the body. The catheter along with the drug-coated or non-coated stent may be sterilized and placed in a catheter package prior to shipping and storing. Additional sterilization using conventional medical means occurs before clinical use. The stent may be coupled to a delivery catheter.

A catheter having a catheter body and an inflation balloon attached to the catheter body near a distal end is inserted into the body, as seen at block 810. The delivery catheter may include an inflatable balloon that is positioned between the stent and the catheter and used for deploying the stent in the body. Alternatively, the delivery catheter may include a sheath that retracts to deploy a self-expanding version of the stent.

The deployment-ready stent is inserted into a vessel of the body, a procedure often performed in a controlled environment such as a catheter lab or hospital. The delivery catheter, which helps position the stent in a vessel of the body, is typically inserted through a small incision of the leg and into the femoral artery, and directed through the vascular system to a desired place in the vessel. Guidewires threaded through an inner lumen of the delivery catheter assist in positioning and orienting the stent. The position of the stent may be monitored, for example, with a fluoroscopic imaging system or an x-ray viewing system in conjunction with radiopaque markers on the stent, radiopaque markers on the delivery catheter, or contrast fluid injected into an inner lumen of the delivery catheter and into an inflatable catheter balloon that is coupled to the stent.

The stent having an optional bioprosthetic valve attached to the stent is delivered and positioned via a catheter to a targeted region within the body. The stent is deployed, for example, by expanding the stent with a balloon or by extracting a sheath that allows a self-expandable stent to enlarge after positioning the stent at a desired location within the body.

After it is positioned, the stent is expanded as seen at block 820. One or more stiffening rings are formed when the stent is expanded and deployed. A bioprosthetic valve that is optionally attached to the stent framework of the stent is deployed in the vessel as the stent is expanded. The formation of stiffening rings as the stent is expanded comprises, for example, substantially straightening a plurality of ring segments connected between circumferentially adjacent crowns of the stent. The stiffening rings are oriented circumferentially about a longitudinal axis of the stent.

When the stent is expanded and deployed, the catheter may be removed from the body, as seen at block 830.

An exemplary procedure employing the present invention is a pulmonic valve replacement. The stent comprises, for example, three stent segments having eight crowns on each side of each stent segment, with stiffening rings on each stent segment and two end segments having no stiffening rings. The stent length is on the order of 24 millimeters, with an expanded or deployed diameter between 18 and 22 millimeters. A stent with an attached one-way bioprosthetic valve such as a bovine jugular valve is positioned between the right ventricle and the pulmonic artery. The pulmonic valve is delivered percutaneously. After suturing the valve to the stent framework, the stent with the valve is positioned over a balloon on a catheter delivery system and crimped or otherwise collapsed onto the inflation balloon. After accessing the body through a femoral vein, the distal end of the catheter is worked up through the inferior vena cava into the right atrium, down into the right ventricle through the ostium into the pulmonary artery. Inflation fluid is injected into the balloon from the proximal end of the delivery catheter and the stent is expanded. When the pulmonic valve is deployed, valve leaflets open and close to allow flow of fluid in the desired direction.

Another exemplary procedure is an aortic valve replacement using a bioprosthetic valve attached to the stent. The stent framework comprises, for example, one stent segment having six crowns per side with a stiffening ring on each end comprised of ring segments connected between circumferentially adjacent crowns. The length is approximately 18 millimeters with a deployed diameter between 18 and 25 millimeters.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, the system comprising:
a catheter; and
a stent coupled to the catheter, the stent including a stent framework having at least one stent segment with a plurality of interconnected struts and crowns and at least one stiffening ring having a plurality of ring segments connected between circumferentially adjacent crowns of the stent segment,
wherein the stiffening ring is oriented circumferentially about a longitudinal axis of the stent framework when the stent is deployed.

2. The system of claim 1, wherein the stiffening ring minimizes overexpansion of the stent framework when the stent is deployed.

3. The system of claim 1, wherein the stiffening ring reduces deployment recoil.

4. The system of claim 1, wherein the catheter includes an inflatable balloon used to expand the stent.

5. The system of claim 1, wherein the catheter includes a sheath that retracts to allow expansion of the stent.

6. The system of claim 1, wherein the stent framework includes at least one end segment having a stiffening ring.

7. The system of claim 1, wherein the stent framework includes at least one end segment having no stiffening ring.

8. The system of claim 1, wherein the stent framework comprises one of a metallic base or a polymeric base.

9. The system of claim 8, wherein the metallic base is selected from the group consisting of stainless steel, nitinol, tantalum, MP35N alloy, a cobalt-based alloy, platinum, titanium, a suitable biocompatible alloy, a suitable biocompatible material, and a combination thereof.

10. The system of claim 1, wherein the stent framework is cut from a tube.

11. The system of claim 1, wherein the crowns of the at least one stent segment are connected to corresponding crowns of an adjacent stent segment with a welded joint.

12. The system of claim 1, wherein the crowns of the at least one stent segment are connected to corresponding crowns of an adjacent stent segment with a molded joint.

13. The system of claim 1, wherein the stent is selected from the group consisting of a cardiovascular stent, a peripheral stent, an abdominal aortic aneurysm stent, a cerebral stent, a carotid stent, an endovascular stent, an aortic valve stent, and a pulmonary valve stent.

14. The system of claim 1, wherein the stent framework has a drug-polymer coating disposed thereon.

15. The system of claim 1 further comprising:
a bioprosthetic valve attached to the stent framework and positioned within a central lumen of the stent framework.

16. The system of claim 15, wherein the bioprosthetic valve comprises a bovine jugular valve.

17. A stent comprising:
a stent framework having at least one stent segment with a plurality of interconnected struts and crowns and at least one stiffening ring having a plurality of ring segments connected between circumferentially adjacent crowns of the stent segment,
wherein the stiffening ring is oriented circumferentially about a longitudinal axis of the stent framework when the stent is deployed.

18. The stent of claim 17, wherein the stent framework has a drug-polymer coating disposed thereon.

19. The stent of claim 17 further comprising:
a bioprosthetic valve attached to the stent framework and positioned within a central lumen of the stent framework.

20. A method of treating a vascular condition, the method comprising:
delivering a stent having a bioprosthetic valve to a targeted region via a catheter;
expanding the stent to deploy the bioprosthetic valve; and
forming at least one stiffening ring of the stent as the stent is expanded.

21. The method of claim 20, wherein forming the at least one stiffening ring as the stent is expanded comprises substantially straightening a plurality of ring segments connected between circumferentially adjacent crowns of the stent, the stiffening ring oriented circumferentially about a longitudinal axis of the stent.
